(19) United States
(12) Patent Application Publication
(10) Pub. No.: US 2006/0068360 A1
(43) Pub. Date: Mar. 30, 2006

(54) Single use fluid reservoir for an endoscope

(75) Inventor: Dennis R. Boulais, Danielson, CT (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignees: SCIMED Life Systems, Inc., Maple Grove, MN (US); Boston Scientific Scimed, Inc.

(21) Appl. No.: 10/955,906

(22) Filed: Sep. 30, 2004

(51) Int. Cl. A61J 7/00; A61C 17/02; A61M 5/31; A61C 17/00; A61M 5/00

(52) U.S. Cl. 433/80; 604/240; 604/77

(57) ABSTRACT

The present invention comprises a single use fluid reservoir for use with a disposable medical device. The single use fluid reservoir has a reservoir body that includes a fluid holding tank. The fluid holding tank includes at least one inlet to admit fluid and at least one outlet to dispense fluid. The fluid reservoir body also has a structure for fixedly connecting the reservoir to the disposable medical device. In some embodiments, the fluid reservoir body has a snap-together structure that is capable of permanently connecting the fluid reservoir to a proximal connector of an endoscope. The fluid reservoir may include a mechanism for alerting an operator to the amount of liquid in the reservoir.

### FIELD OF THE INVENTION

The present invention relates to single use medical devices in general and a single use fluid reservoir for a single use endoscope in particular.

### BACKGROUND OF THE INVENTION

It has become well established that there are major public health benefits from regular endoscopic examinations of a patient's internal structures such as the alimentary canals and airways, e.g., the colon, esophagus, stomach, lungs, uterus, urethra, kidney and other internal organ systems. Conventional imaging endoscopes used for such procedures generally comprise a flexible tube with a fiber optic light guide that directs illuminating light from an external light source to the distal tip where it exits the endoscope and illuminates the region to be examined. An objective lens and fiber optic imaging light guide communicating with a camera at the proximal end of the scope, or an imaging camera chip at the distal tip, produce an image that is displayed to the operator. In addition, most endoscopes include one or more working channels through which medical devices such as biopsy forceps, snares, fulguration probes, and other tools may be passed. Water can be selectively applied to a tube that is connected to a working channel of the endoscope to clean the working channel. Water may also be used to irrigate the patient. Sterilized water is provided from a fluid reservoir. The fluid lines and fluid reservoirs are cleaned between procedures to prevent cross-contamination.

Conventional endoscopes are expensive hand assembled medical devices costing in the range of $25,000 for an endoscope, and much more for the associated operator console. Because of the expense, these endoscopes are built to withstand repeated disinfections and use upon many patients. Conventional endoscopes are generally built of sturdy materials, which decreases the flexibility of the scope and thus can decrease patient comfort. Furthermore, conventional endoscopes are complex and fragile instruments that frequently need expensive repair as a result of damage during use or during a disinfection procedure.

Low cost, disposable medical devices designated for a single use have become popular for instruments that are difficult to sterilize or clean properly. Single use, disposable devices are packaged in sterile wrappers to avoid the risk of pathogenic cross-contamination of diseases such as HIV, hepatitis and other pathogens. Hospitals generally welcome the convenience of single use disposable products because they no longer have to be concerned with product age, overuse, breakage, malfunction and sterilization. One medical device that has not previously been inexpensive enough to be considered truly disposable is the endoscope, such as a colonoscope, bronchoscope, gastroscope, duodenoscope, etc. Such an endoscope is described in U.S. patent application Ser. No. 10/811,781, filed Mar. 29, 2004, assigned to Scimed Life Systems, Inc., the assignee of the present invention, and in a U.S. Continuation-in-Part Patent Application filed Sep. 30, 2004, and identified by Attorney Docket No. BSEN123550, which are herein incorporated by reference.

While the endoscope disclosed in U.S. patent application Ser. No. 10/811,781, and in the Continuation-in-Part application filed Sep. 30, 2004, and identified as BSEN123550, reduces the risk of cross-contamination from the endoscope itself, there remains a risk that the fluid reservoir that supplies the endoscope will be improperly used for multiple endoscopic procedures. To limit the chance of such unauthorized reuse, there is a need for a single use fluid reservoir and a system to prevent the re-use of a single use fluid reservoir with a single use endoscope system.

### SUMMARY OF THE INVENTION

To address these and other problems in the prior art, the present invention is a single use fluid reservoir for use with an endoscope system. The single use fluid reservoir includes a fluid holding tank having at least one inlet to admit fluid and at least one outlet to dispense fluid. The fluid reservoir body also has a structure for connecting to a disposable endoscope. In some embodiments, the fluid reservoir body has a snap-together structure that is capable of permanently connecting the single use fluid reservoir to the disposable endoscope.

In another aspect, the present invention is a single use fluid reservoir system that prevents reuse. The system comprises a single use fluid reservoir that includes a fluid holding tank with at least one inlet to admit fluid and at least one outlet to dispense fluid, and a connecting structure on the fluid reservoir adapted to fixedly connect to a proximal connector of a single use endoscope. The system also includes a proximal connector on a single use endoscope having a connecting structure adapted to fixedly connect to the single use fluid reservoir. In operation, the single use fluid reservoir and the proximal connector on the single use endoscope are attached together prior to clinical use to form a disposable unit that is disposed of after use. In some embodiments, the single use fluid reservoir further comprises a fluid level detection element. The single use reservoir of the invention is useful in the single use reservoir system of the invention.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic diagram of an endoscope system comprising a single use fluid reservoir connected to a proximal connector of the endoscope in accordance with an embodiment of the present invention;

FIG. 2 is cross-sectional view of a representative embodiment of a single use fluid reservoir attached to a proximal connector to form a disposable unit in accordance with an embodiment of the present invention;

FIG. 3 is a perspective view of a representative embodiment of a single use fluid reservoir;

FIG. 4 is a perspective view showing a representative set of corresponding snap-together structures on a single use fluid reservoir and on a proximal connector in accordance with one embodiment of the present invention;

FIG. 5 is a perspective view showing a single use fluid reservoir connected to a proximal connector via snap-together structures in accordance with an embodiment of the present invention;

FIG. 6 is a cross-sectional view of a single use fluid reservoir and proximal connector shown in FIG. 5 showing snap-together points of attachment;

FIG. 7 is a perspective view of an embodiment of a single use fluid reservoir having a fluid sight window in accordance with an embodiment of the present invention;

FIG. 8 is a diagram illustrating an embodiment of a single use fluid reservoir having a fluid detection system comprising a float ball in accordance with an embodiment of the present invention;

FIG. 9 is a perspective view of an embodiment of a single use fluid reservoir having a fluid sensor rod in accordance with an embodiment of the present invention;

FIG. 10 is a cross-sectional view of an embodiment of a single use fluid reservoir having a floating dipstick integrated into a removable cap in accordance with an embodiment of the present invention; and

FIG. 11 is a cross-sectional view of an embodiment of a single use fluid reservoir having a sensor cap with an integrated reusable sensor in accordance with an embodiment of the present invention.

### DETAILED DESCRIPTION OF THE PREFERRED EMBODIMENT

In traditional endoscopic systems, fluid is used for various purposes such as to irrigate tissue in a patient, flush away debris from the lens, etc. Sterilized fluid is provided through fluid lines to the endoscope from a fluid reservoir. In traditional endoscope systems, the fluid lines and fluid reservoirs are cleaned between procedures to prevent cross-contamination from one patient to another. As discussed above, with the emergence of disposable medical devices, the use of devices packaged in sterile wrappers decreases the risk of cross-contamination of diseases such as HIV, hepatitis and other pathogens. However, the fluid reservoir and fluid lines are often sterilized and reused, creating a potential source of cross-contamination.

The present invention provides a fluid reservoir system that is designed to be disposable and difficult to reuse and thereby reduces the likelihood of cross-contamination of fluids in an endoscope system. Generally described, the single use fluid reservoir includes a fluid holding tank having at least one fluid inlet and at least one fluid outlet. The fluid reservoir also includes a structure for fixedly connecting the fluid reservoir to a portion of the disposable endoscope, such as a proximal connector, which in turn is capable of connecting the disposable endoscope to a control unit.

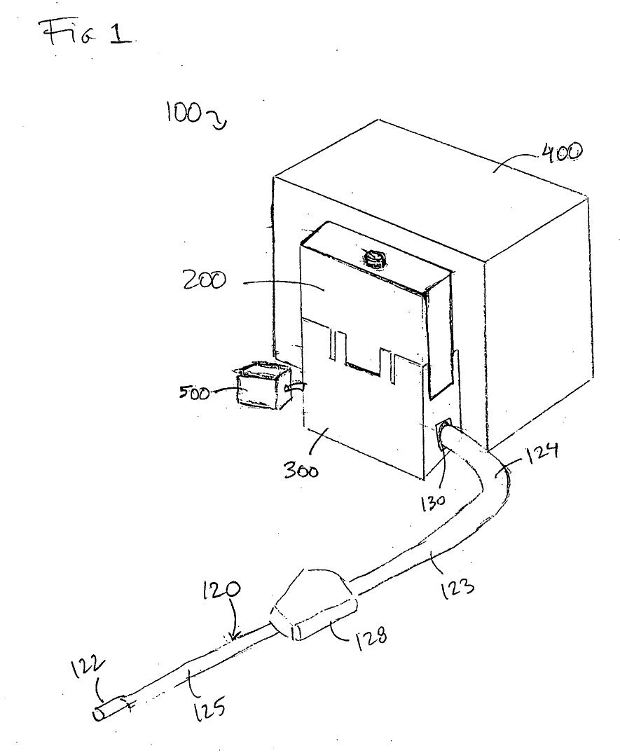

FIG. 1 illustrates the components of an exemplary endoscope system 100 having a single use fluid reservoir 200 according to one embodiment of the present invention. The major components of the endoscope system 100 include an endoscope 120, a single use fluid reservoir 200 that is attached to a proximal connector 300 of the endoscope that is in turn removably connected to a reusable control unit 400. The endoscope 120 comprises a shaft 123 having a distal end 125 and a proximal end 124. The distal end 125 includes a tip 122 having an imaging element (not shown) and the proximal end 124 has a connector 130 that attaches (i.e. removably) the endoscope to the connector 300.

In the embodiment shown, the endoscope 120 also includes a breakout box 128 that is positioned approximately midway along the length of the shaft 123. The breakout box 128 provides an entrance to a working channel and may include additional attachment points for collection of samples and surgical manipulation. As shown, the single use fluid reservoir 200 is securely attached to the proximal connector 300 of the endoscope. A pump 500 is removably attached to a section of tubing protruding from inside the proximal connector 300. While the illustrative embodiment of the endoscope system 100 having a single use fluid reservoir 200 depicted in FIG. 1 shows an endoscope as the single-use device, it will be understood by one skilled in the art that any type of single use medical device that requires a source of fluid can be used in accordance with the single use fluid reservoir 200. For example, the present invention may be used in connection with dental devices that are used once and disposed of, such as suction tubes.

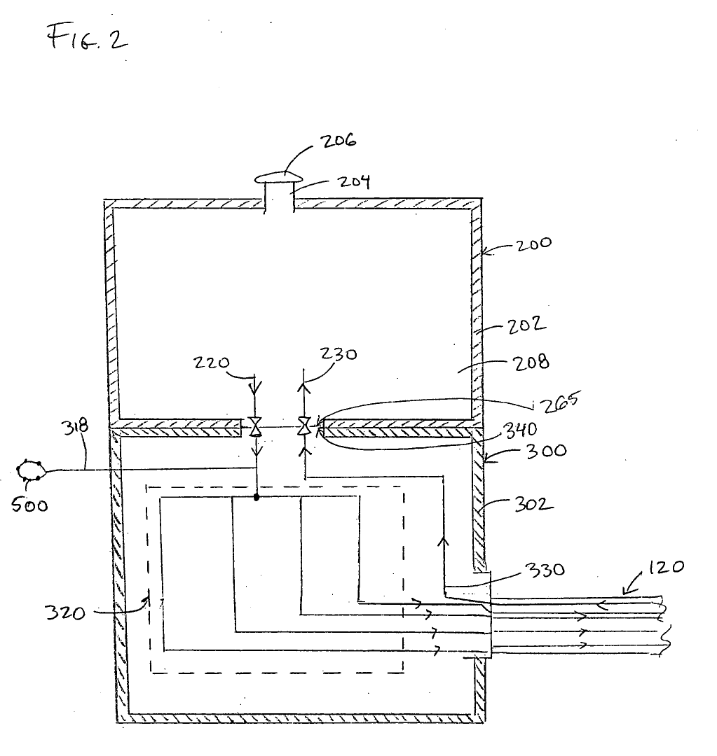

FIG. 2 is a cross-sectional view of a representative embodiment of the single use fluid reservoir 200 connected to the proximal connector 300 of an endoscope. As shown, the fluid reservoir 200 has an outer housing 202 that surrounds or itself forms an inner fluid holding tank 208. A fill spout 204 in fluid communication with the fluid holding tank 208 is positioned on the top of the fluid reservoir and is shown covered with a removable cap 206.

In the embodiment shown in FIG. 2, the single use fluid reservoir 200 is attached to the proximal connector 300 via a fluid reservoir connector 265 and a proximal connector receptacle 340 that provides a fluid connection between an outlet tube 220 emerging from the fluid holding tank 208 and a fluid inlet tube 318 in the proximal connector 300. The fluid inlet tube 318 is further attached to a pump 500 that is capable of drawing fluid from the fluid holding tank 208 into tubing in a fluidics manifold 320 contained in the proximal connector 300. The pump 500 is preferably a peristaltic pump so that the pump components do not come into contact with the fluid; therefore the pump 500 may be reused without a risk of fluid cross-contamination.

As shown in FIG. 2, the fluidics manifold 320 in the proximal connector 300 allows fluid to flow in selected directions for various functions of the endoscope 120. An example of a fluidics manifold suitable for use in a single use proximal connector is described in more detail in U.S. patent application Ser. No. 10/811,781 filed Mar. 29, 2004, and in the Continuation-in-Part application filed Sep. 30, 2004, and identified as BSEN123550, mentioned above. Fluid flow through each path in the fluidics manifold 320 is selectively controlled with valves (not shown) housed in the control unit 400 attached to the proximal connector 300. Pinch valves in the control unit 400 are preferred because the valve components do not need to come into contact with the fluids within the endoscope system, therefore the control unit 400 may be reused without a risk of cross-contamination.

In some embodiments of the endoscope system 100, fluid is continually pumped through a heat exchanger in the distal tip 122 of the endoscope 120 in order to prevent illumination sources, such as LEDs from becoming too hot in a patient's body. As shown in FIG. 2, fluid is delivered via tubing from the fluid manifold 320 to the endoscope 120 where the fluid is carried to a heat exchanger that is thermally coupled to an LED illumination source (not shown) at the distal end of the endoscope 120. Fluid returning from the heat exchanger is carried back through the endoscope 120 and is received into a tube 330 in the proximal connector 300 and in turn is passed back through a tube 230 to the fluid holding tank 208 for fluid recirculation.

Fluid may also be supplied from the single use fluid reservoir 200 to the manifold 320 for additional functions in the endoscope system 100. For example, fluid may be selectively applied to a tube that provides a high pressure lavage for irrigating a patient lumen, as well as to a lens wash tube that cleans contaminants from the front of an imaging lens at the distal end 122 of the endoscope 120. Fluid may also be selectively applied to a tube that is connected to a working channel tube of the endoscope to clean the working channel. Fluid that comes into direct contact with patient tissue is generally removed by vacuum aspiration to a collection jar and is not returned to the reservoir.

In accordance with this aspect of the invention, the single use fluid reservoir 200 contains connection elements that securely and non-removably attach it to a point on the endoscope, such as the proximal connector 300 so that once attached, the fluid reservoir and the endoscope form a connected, disposable unit. A secure, non-removable attachment between the fluid reservoir and the endoscope may be formed using any combination of connectors suitable to provide a functional and secure connection such as, for example, a set of one-way snap-together elements. The connecting structures may be formed as a unitary portion with the housing of the fluid reservoir and proximal connector of the endoscope. Alternatively, the connecting structures may be formed separately and connected to the housing of the fluid reservoir and proximal connector. The proximal connector 300 and fluid reservoir 200 could also be molded together.

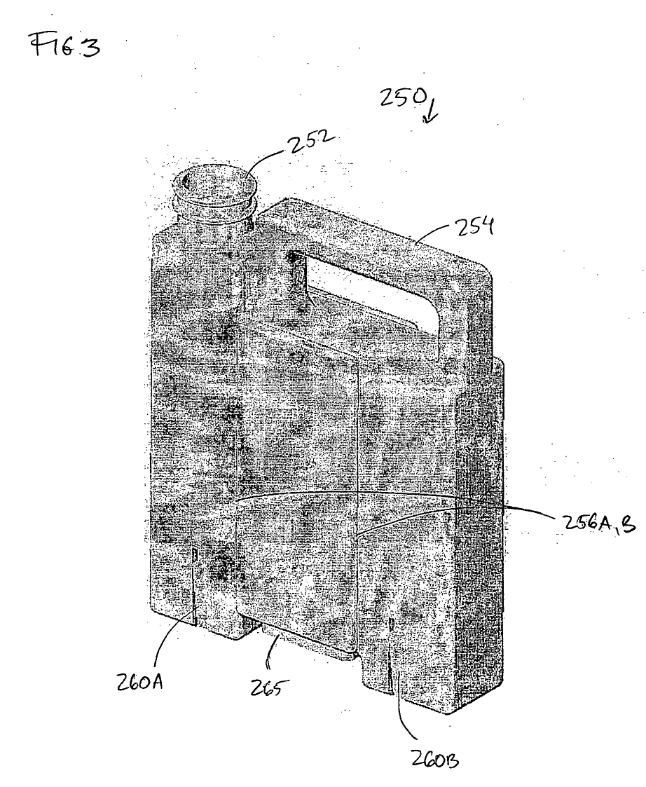

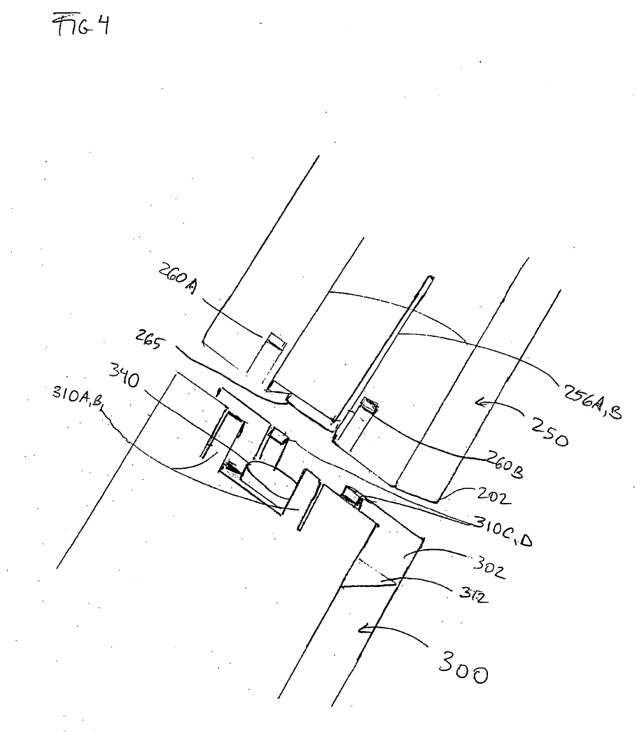

FIG. 3 illustrates a representative embodiment of a single use fluid reservoir 250 capable of forming a secure, non-removable connection with an endoscope. As shown, the fluid reservoir 250 has a fill spout 252 and a handle 254 on the upper end portion of the fluid reservoir body, and a connector 265 at the lower end portion to achieve a fluid connection with a proximal connector. A set of retention pockets 260A, 260B are capable of connecting to a corresponding set of latch arms on the proximal connector 300 (FIG. 4). The retention pockets 260A, 260B are molded into the housing of the fluid reservoir 200 as shown more clearly in FIG. 4.

FIG. 4 illustrates a set of representative inter-connecting structures capable of locking the embodiment of the fluid reservoir 250 shown in FIG. 3 to the proximal connector 300. As shown in FIG. 4, the lower end portion of the fluid reservoir 250 is sized in relation to the upper end portion of the proximal connector 300 such that the lower end portion of the fluid reservoir 250 fits snugly against the upper end portion of the proximal connector 300. As further shown in FIG. 4, a set of inter-connecting structures are formed in the housing of the fluid reservoir 202 in the form of four retention pockets, 260A,B (C, D not shown). The housing of the proximal connector 302 include four corresponding latch arms 310A, B, C, D for securely connecting the fluid reservoir 250 to the proximal connector 300. A platform 312 on the upper end portion of the proximal connector 300 provides a surface upon which the lower end portion of the fluid reservoir 250 rests. The fluid reservoir connector 265 attaches to a proximal connector receptacle 340, thereby allowing fluid connection between tubing in the fluid reservoir tank and the tubing connected to a manifold in the proximal connector as described in more detail above in reference to FIG. 2.

The latch arms 310A-D and the corresponding retention pockets 260A, B (C, D not shown) are preferably spaced apart from one another to provide a secure, locking connection between the fluid reservoir 250 and the proximal connector 300. The width of the latch arms 310A-D matches the width of the retention pockets 260A-D. In the embodiment shown in FIG. 4, the fluid reservoir 250 additionally has at least two guide rib details 256A,B cut into the outer reservoir housing 202, to enable correct positioning of the reservoir 250 over the proximal connector 300 during attachment.

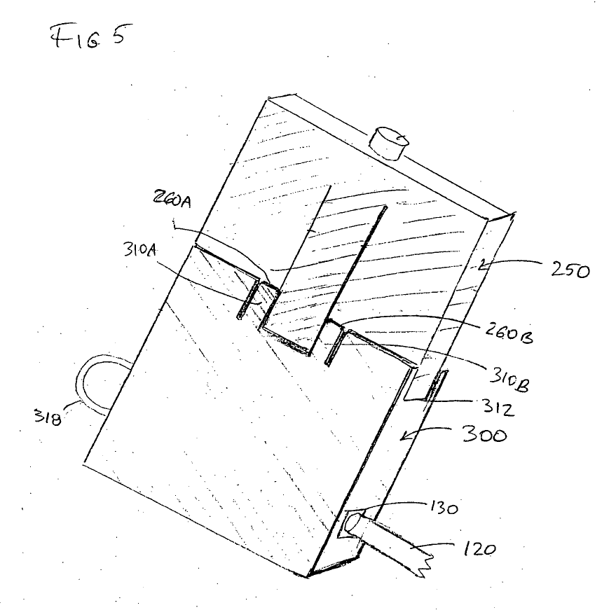

FIG. 5 is a perspective view showing the embodiment of the single use fluid reservoir 250 illustrated in FIG. 4 attached to the proximal connector 300 via the snap-together connecting structures to form a single disposable unit. As shown in FIG. 5, the lower end portion of the fluid reservoir 250 fits snugly within the recess 312 in the upper end portion of the proximal connector 300. The latch arms 310A-D on the proximal connector 300 are shown snapped into the retention pockets 260A-D in the single use fluid reservoir housing, resulting in a secured, non-removable connection. A loop of tubing 318 is shown protruding from the proximal connector 300 that is designed to be inserted into the pump mechanism 500 so that fluid may be pumped from the fluid reservoir 250 into the proximal connector 300.

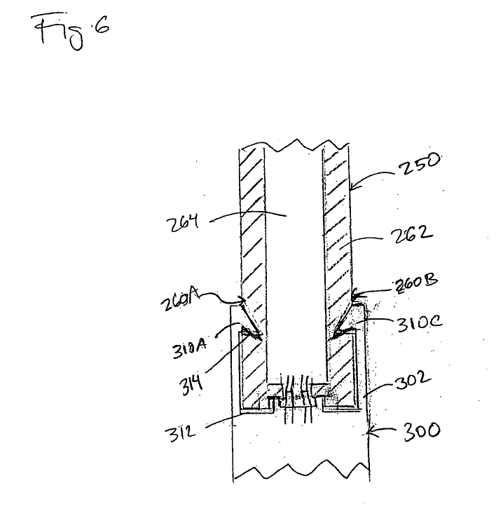

FIG. 6 shows a cross-sectional view of the cooperating latch arms, e.g., 310A, 310B, 310C, 310D within the corresponding retention pockets 260A, 260B, etc. The latch arms 310A,C (B,D are not shown) have angled or barbed locking heads 314 that engage the retention pockets 260A-D to snap-together the fluid reservoir 250 and the proximal connector 300. The latch arms 310A-D are preferably elastically deformable or flexible such that the lock heads 314 are able to deflect radially outwardly from the central axis of the proximal connector 300 upon the force of the fluid reservoir being inserted and return to their original condition upon removal of the force in order to engage the retention pockets 260A-D. The latch arms 310A-D may be formed as a flexible portion with the proximal connector body 302 out of a structural plastic material such as nylon, glass-filled nylon or the like. Alternatively, one or both connecting structures may be made separately and then joined to the reservoir or connector either following formation or integrated therewith during manufacture (e.g., molded in place).

In operation of the endoscope system 100, with reference to FIG. 1, the proximal connector 300 and endoscope 120 are removably attached to the control unit 400. The single use fluid reservoir 200 is securely and non-removably attached to the proximal connector 300, forming a single disposable unit. The fluid reservoir 200 may be filled with fluid prior to, or after, attachment to the proximal connector 300. After a single clinical use, the disposable unit comprising the single use reservoir 200, proximal connector 300, and the endoscope 120 is disconnected from the control unit 400 and discarded, thereby preventing potential reuse.

In some instances, especially during complex or lengthy procedures, an additional amount of fluid may be needed to supplement the fluid in the fluid holding tank. In reference to FIG. 2, the fluid holding tank 208 may be refilled via the fill spout 204 after the fluid reservoir 200 is connected to the proximal connector 300. During such a lengthy procedure, an operator would preferably have a means by which to monitor the fluid level in the fluid holding tank 208. Accordingly, in some embodiments, the single use fluid reservoir further includes a fluid level detection element. Preferably, the fluid level is detected using a disposable, low cost method of detection. In alternative embodiments, the fluid level detection element is a reusable sensor inserted into the disposable single use fluid reservoir via the fill spout 204.

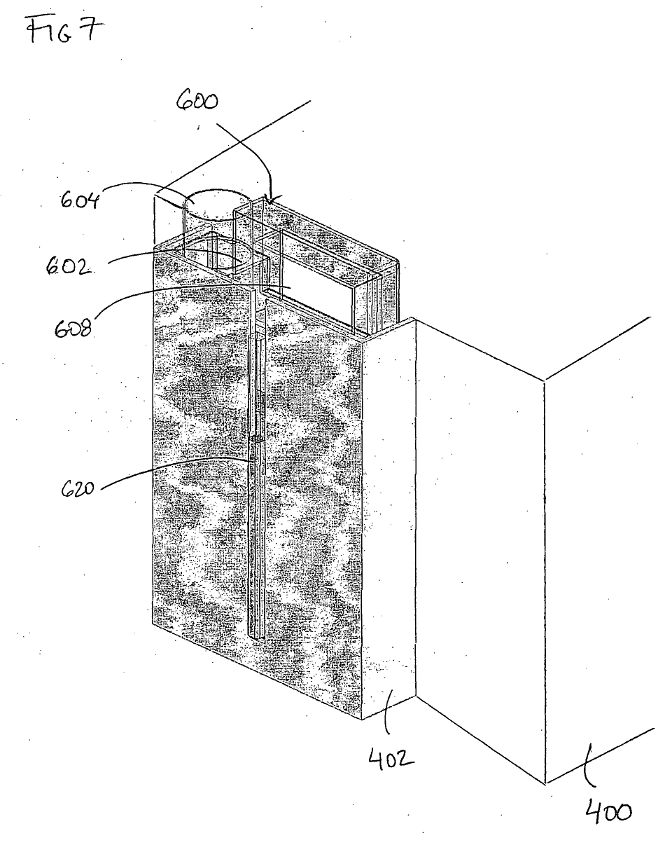

FIG. 7 illustrates one embodiment of a single use fluid reservoir 600 having an integrated fluid detection element according to the present invention. As shown, a fluid sight window 620 is provided along one side of a fluid reservoir tank 608. In the embodiment shown in FIG. 7, an optional console door 402 is removably attached to the control unit 400 and fitted over the single use fluid reservoir 600 when it is attached to the proximal connector (covered by the door 402). The fluid sight window 620 protrudes through a slot in the console door 402, thereby allowing an operator to visibly determine the fluid level in the fluid reservoir 600. In the embodiment shown, the fluid sight window 620 is integrally formed with the housing of the fluid reservoir. For example, the housing of the fluid reservoir may be made of a clear material, such as glass or clear plastic. Alternatively, the reservoir may be made of an opaque material and the fluid sight window 620 may be formed out of a clear material and attached to a slot formed along one side of the reservoir 600. In operation, the operator visually monitors the fluid sight window 620 during an endoscope procedure and refills the fluid reservoir tank 608 through the fill spout 604 when the fluid level drops below a desired level.

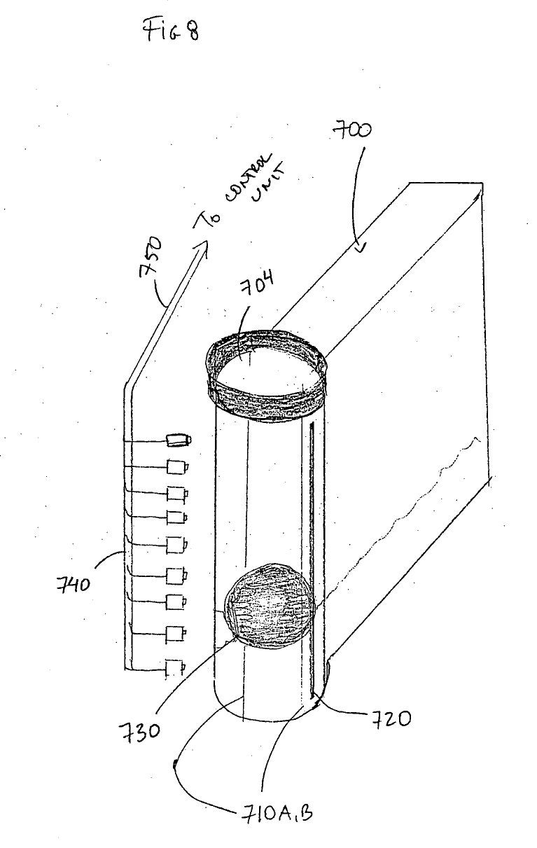

FIG. 8 shows another alternative embodiment of a single use fluid reservoir 700 having an integrated fluid detection element according to the present invention. In this embodiment, a single use fluid reservoir 700 has a housing and fluid holding tank formed from a clear material. The fluid holding tank contains a float ball 730 that is sized such that it is confined in a column formed by two ribs 710A,B included in the walls of the fluid holding tank. The float ball 730 floats on the surface of the fluid in the reservoir and is allowed to move vertically up and down in the column of the fluid holding tank as the fluid level changes. A reflective strip 720 is attached in a vertical line to one side of the fluid reservoir 700. On the side of the fluid reservoir opposite the reflective strip 720 is attached a series of optical or other sensors 740 that are arranged in a vertical line. The sensors 740 are preferably arranged so that the float ball 730 is always blocking one of the beams from one of the series of sensors 740. A signal cord 750 attached to the optical sensors 740 carries the electrical signal from the optical sensors to a control unit to produce an output display (not shown) indicating the level of fluid in the fluid reservoir 700.

In operation, light from the optical sensors 740 passes through the reservoir tank column and hits the reflective strip 720. The light then passes back through the reservoir tank to receivers in the optical sensors. The float ball 730 rides on the surface of the fluid inside the column in the fluid holding tank. As the fluid level changes, the float ball 730 blocks the reflected light from reaching one or more of the optical sensors 740. The sensor signals are sent via the cable 750 to a control unit (not shown) which provides a suitable visual or audio output indicating the fluid level to an operator.

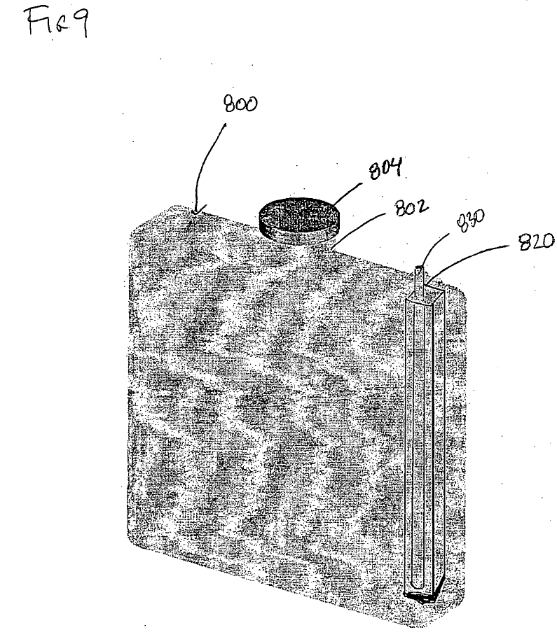

FIG. 9 shows another alternative embodiment of a single use fluid reservoir 800 having an integrated fluid sensor element. As shown, the single use fluid reservoir 800 is formed with a sensor rod 830 surrounded by a protective casing 820 integrated into the body of the fluid reservoir 800. The sensor rod 830 may be any single use type of sensor and may be disposed of along with the single use fluid reservoir 800. Alternatively, the sensor rod 830 may be removable from the protective casing 820, thereby allowing the use of a reusable sensor that does not contact the fluid in the fluid holding tank in order to prevent cross-contamination. As an illustrative example, the sensor rod 830 may be a single use or reusable metal rod that allows for fluid level detection via a capacitive sensor positioned along one side of the fluid reservoir. The capacitive sensor is a non-contact device that uses capacitive sensing technology to detect the level of fluid in the fluid holding tank. Such a sensor may be a continuous level sensor or a point level sensor. Briefly described, a capacitive sensor includes an electrode assembly, an oscillator circuit and an output circuit. The electrode assembly is designed so that an electrostatic field is formed between an active electrode and an earth electrode. Any object (e.g. fluid) entering this field will increase the capacitance as a function of the dielectric constant of the material and the amount of target material. The increase in capacitance sets up an oscillation that changes the state of the output circuit. Capacitive sensors suitable for use with the single use fluid reservoir 800 are commercially available (e.g., from IFM Efector, Inc. Exton Pa.).

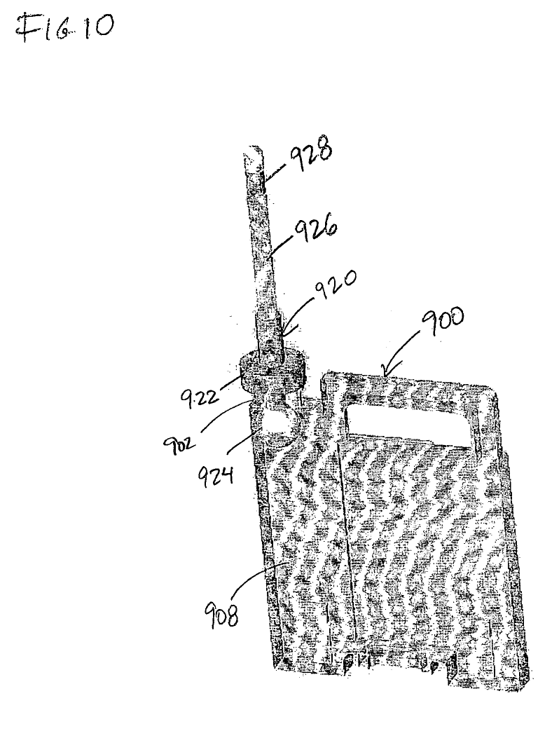

FIG. 10 shows a cross-sectional view of another alternative embodiment of a single use reservoir 900 having a disposable sensor element integrated into a removable cap connected to the fluid reservoir. As shown, a single use fluid reservoir 900, similar to the embodiment shown in FIG. 3, has a fill spout 902 into which a floating dipstick 920 is inserted via a removable cap 922. The floating dipstick 920 has a ball float 924 connected to a hollow shaft 926 which protrudes through the removable cap 922. At or near the top of the shaft 926 of the floating dipstick 920 is an indicator flag 928 that indicates the fluid level in the fluid holding tank 908. The indicator flag 928 may be brightly colored, and/or made out of metal to allow detection via a capacitive sensor positioned on a control unit. In operation, an operator may visually determine that the fluid level in the tank 908 is below a desired level when the indicator flag 928 drops below a designated position. Alternatively, if the fluid level is detected via a capacitive sensor, the operator may view an output display on the control unit.

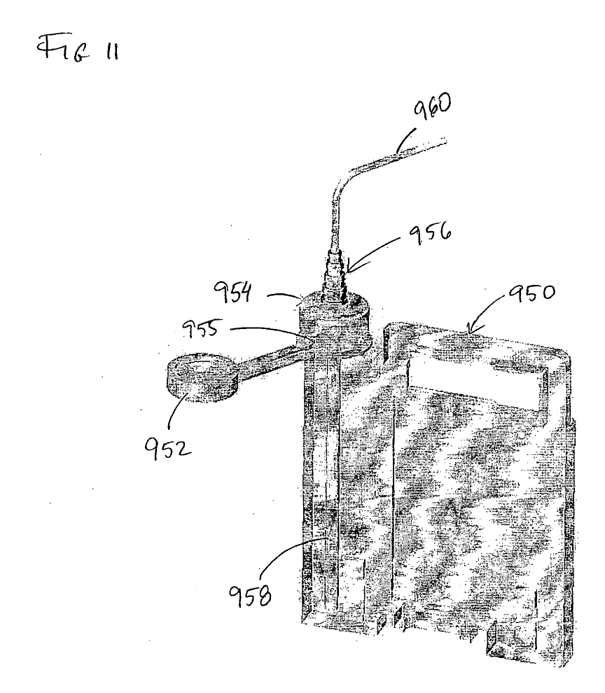

In an alternative embodiment, the present invention provides a single use fluid reservoir 950 having a reusable fluid sensor element. FIG. 11 shows a cross-sectional view of a representative fluid reservoir 950 according to this embodiment of the invention. As shown, a temporary cap 952 is removably attached to the fluid reservoir 950. A second removable sensor cap 954 having an integrated fluid sensor element 956 is shown inserted into a fill spout 955. The fluid sensor 956 has a submersible sensor element 958 attached below the sensor cap 954 and a sensor signal cable 960 that carries a fluid level output signal to a control unit (not shown). The fluid level sensor 956 may comprise any reusable sensor element capable of detecting the fluid level in the tank, such as for example, a capacitive sensor, a pressure sensor, an optical sensor, an ultra-sonic sensor, or a radar sensor, etc. In operation, an operator fills the fluid reservoir with fluid and closes the temporary cap 952 prior to starting the endoscope procedure. The fluid reservoir may be connected to the proximal connector either prior to, or after filling the reservoir tank with fluid. In preparation for the endoscope procedure, the operator removes the temporary cap 952 and replaces it with the sensor cap 954. The signals from the fluid sensor 956 are then included in an output display, typically connected to a control unit. Alternatively, the sensor signal can be used to trigger an audible or visual alarm if the fluid level gets too low. Once the endoscope procedure is completed, the sensor cap 954 is removed from the reservoir tank and the sensor 958 is cleaned between procedures. The fluid reservoir 950 attached to the connector is thrown away as a disposable unit.

While the preferred embodiment of the invention has been illustrated and described, it will be appreciated that various changes can be made therein without departing from the scope of the invention. It is therefore intended that the scope of the invention be determined from the following claims and equivalents thereof.

The embodiments of the invention in which an exclusive property or privilege is claimed are defined as follows:

1. A single use fluid reservoir for use in an endoscope system, the single use fluid reservoir comprising:
a fluid reservoir body that includes a fluid holding tank, wherein the fluid holding tank has at least one inlet to admit fluid and at least one outlet to dispense fluid; and
means for fixedly connecting the fluid reservoir body to an endoscope such that the endoscope and fluid reservoir can be disposed of as a unit.

2. The single use fluid reservoir of claim 1, wherein the fluid reservoir is fixedly connected to a proximal connector of an endoscope and the means for connecting the fluid reservoir to the proximal connector comprises one or more flexible snap-together structures.

3. The single use fluid reservoir of claim 2, wherein the one or more snap-together structures include at least one retention pocket and a corresponding latch arm that fits within the retention pocket.

4. The single use fluid reservoir of claim 3, further comprising at least two guide ribs on the reservoir body capable of aligning the at least one retention pocket with a latch arm.

5. The single use fluid reservoir of claim 1, further comprising a fluid level detection element.

6. The single use fluid reservoir of claim 5, wherein the fluid level detection element is a fluid sight window.

7. The single use fluid reservoir of claim 5, wherein the fluid level detection element comprises a buoyant member with sensor means for detecting the buoyant member.

8. The single use fluid reservoir of claim 5, wherein the fluid level detection element comprises a submersible sensor element.

9. The single use fluid reservoir of claim 8, wherein the submersible sensor element is removably attached to the fluid reservoir.

10. A single use fluid reservoir system that prevents reuse, the system comprising:
a single use fluid reservoir including a fluid holding tank, wherein the fluid holding tank includes at least one inlet to admit fluid and at least one outlet to dispense fluid, and a connecting structure adapted to fixedly connect to a proximal connector of an endoscope.

11. The system of claim 10, wherein the single use proximal connector has a body with a first connecting structure adapted to fixedly connect to the single use fluid reservoir, and a second connecting structure adapted to removably connect the proximal connector to a control unit for the endoscope.

12. The system of claim 10, wherein the single use fluid reservoir and the single use proximal connector are permanently attached together prior to clinical use to form a disposable unit.

13. The system of claim 10, wherein the disposable unit further comprises a fluid level detection element.

14. The system of claim 13, wherein the fluid level detection element is a fluid sight window.

15. The system of claim 13, wherein the fluid level detection element comprises a buoyant member with sensor means for detecting the buoyant member.

16. The system of claim 13, wherein the fluid level detection element comprises a submersible sensor element.

17. The system of claim 13, wherein the submersible sensor element is removably attached to the fluid reservoir.
